(19) United States
(12) Patent Application Publication
(10) Pub. No.: US 2011/0183904 A1
(43) Pub. Date: Jul. 28, 2011

(54) METHODS AND COMPOSITIONS FOR ALTERING BEHAVIOR ASSOCIATED WITH AUTISM SPECTRUM DISORDER

(75) Inventors: Jill Jacobson, Kansas City, MO (US); Kathryn Ellerbeck, Kansas City, KS (US)

(73) Assignee: The Children's Mercy Hospital, Kansas City, MO (US)

(21) Appl. No.: 12/937,526

(22) PCT Filed: Apr. 10, 2009
(86) PCT No.: PCT/US09/40282

§ 371 Date: Dec. 30, 2010

Related U.S. Application Data
(63) Continuation of application No. 61/044,049, filed on Apr. 10, 2008.

(51) Int. Cl. A61K 38/11; G01N 33/68; A61K 31/55; A61P 43/00

(52) U.S. Cl. 514/10.9; 436/86; 514/11.6; 514/213.01

(57) ABSTRACT

The present invention discloses methods for the diagnosis and treatment of autism. The diagnostic tools provided use analysis of levels of G-protein expression, oxytocin, and vasopressin. Treatment for autism spectrum disorder and symptoms of autism spectrum disorder, especially repetitive behavior, is also provided.

### RELATED APPLICATIONS

This application claims the priority benefit of Provisional Application Ser. No. 61/044,049, filed Apr. 10, 2008. The teachings and content of this priority application are hereby incorporated by reference in their entireties.

### SEQUENCE LISTING

A sequence listing in electronic format is being filed together with the application. The content of this sequence listing is incorporated by reference in its entirety.

### BACKGROUND AND HISTORY OF THE PRIOR ART

Autism Spectrum Disorders (ASD), also known as Pervasive Developmental Disorders (PDDs), cause severe and pervasive impairment in thinking, feeling, language, and the ability to relate to others. These disorders are usually first diagnosed in early childhood and range from a severe form, called autistic disorder, through pervasive development disorder not otherwise specified (PDD-NOS), to a much milder form, known as Asperger syndrome. They also include two rare disorders, Rett syndrome and childhood disintegrative disorder. Children with ASD are typically unresponsive to people and do not appear to focus intently on one item for long periods of time. The first signs of an ASD can appear in a child who had been developing normally but becomes silent, withdrawn, self-abusive, or indifferent to social overtures.

Previous studies indicated that low levels of G-proteins may be responsible for other neurological disorders. It was thought that these low levels of G-proteins contributed to the outwards symptoms of the disorders, but these G-protein levels have not previously been analyzed in regard to autism. Autism is becoming an epidemic with more cases being diagnosed every day. More sophisticated diagnostic testing is needed in order to properly treat children on the autism scale and provide more individualized diagnosis and treatment.

### SUMMARY OF THE INVENTION

The present invention is directed towards diagnostic techniques and treatment for ASD. The present invention is based on the finding that levels of O-proteins play a role in Autism Spectrum disorders (ASD). Specifically, it was found that levels of G-proteins are higher in children diagnosed with ASD than those children who do not exhibit any symptoms. Further, it was discovered that the G-proteins specific for ASD are preferably, Gαs and Gαq. This is the first disclosure that Gαs levels are elevated in PBMCs of children with ASD.

Cells rely on a relay system to get messages from the outside to the inside of the cell so that the appropriate biological response can take place. These messages include neurotransmitters and hormones and include dopamine, serotonin, and oxytocin. The middleman in 80% of the relay systems is the G-protein. There are several types of G-proteins, but for purposes of the present invention, two G-proteins, Gαs and Gαq, are preferred.

As is known in the art, messages from outside of a cell, e.g. from dopamine, bind to receptors on the surface of cells. The G-protein (GTP) within the cell senses this action and that there is a message to send, so it gives part of itself to “relay” the message on and becomes GDP. Through the G-protein passing on the message, the inside of the cell now knows what it is supposed to do.

G-proteins are thought to be important to behavioral disorders and mental illnesses. Recent research has shown that a range of behaviors actually relies on second messenger signals within cells. Increased Gαs in a particular type of white blood cell called a polymorphonuclear cell (PBMC) has been found in neuropsychiatric conditions, including bipolar illness and schizophrenia. In both bipolar illness and schizophrenia, treatment with medications such as lithium or haldol has been shown to decrease Gαs levels in PBMCs. It was found that, similar to the findings in bipolar illness and schizophrenia, children with autism have significantly higher Gαs levels than did typically developing children. It was also found that boys with autism had higher plasma levels of oxytocin than typically developing controls and girls with autism. This is the first disclosure showing that Gαs levels in PBMCs are elevated in children with ASD, as this has not previously been reported. Additionally, malfunctioning of G-protein signaling has not received much attention in children with ASD, and increased Gαs levels in PBMCs in children with autism have not been previously reported. It was additionally observed that children with ASD showed elevated Gαq levels in PMBCs, as it was found that the correlation coefficient between Gαs and Gαq is 0.96.

Oxytocin (OT) has been implicated in autism because of its role in social affiliation and repetitive behavior. OT is a hormone that uses stimulatory G-proteins as its signal transducer and has been implicated in ASD because it is shown to be correlated with social competence and repetitive motor mannerisms. Heterotrimeric G-proteins transduce signals from over 80% of extracellular signaling molecules, including oxytocin. Oxytocin is a neuropeptide that uses the Gαq subclass of G-proteins. Abnormalities in G-proteins have been proposed to lead to a functional imbalance in multiple neurotransmitter pathways and to immune dysfunction. The inventors previously found that (1) female mice express more Gαq in lymphocytes as compared to males and (2) that more Gαq gene expression results in more oxytocin “action” and a larger proliferative response when immune cells are exposed to oxytocin. Oxytocin has been implicated in autism because of its role in social affiliation and repetitive behavior. Heterotrimeric G-proteins transduce signals from over 80% of extracellular signaling molecules, including oxytocin.

Arginine vasopressin, known as vasopressin, is a peptide hormone found in most mammals, which is derived from a preprohormone precursor that is synthesized in the hypothalamus and stored in vesicles at the posterior pituitary. Vasopressin also signals through G-proteins, and therefore, may play a role in autism. PBMCs were exposed to vasopressin and G-protein levels were analyzed. It was found that exposure to vasopressin had the effect of up-regulating the expression of these G-proteins, specifically Gαs and Gαq in healthy patients. This was not observed in patients with ASD, as their protein expression levels decreased in response to vasopressin exposure.

Another aspect of the present invention involves altering OT levels in order to diminish ASD characteristics. Levels of G-proteins can be altered by either the up-regulation or down-regulation of oxytocin and/or vasopressin levels. In a preferred embodiment, down-regulation of oxytocin and/or vasopressin is achieved through the use of a oxytocin or vasopressin antagonist. Any antagonist of oxytocin and/or vasopressin will work with the methods of the present invention. A preferred antagonist of oxytocin is FE 200 440. A preferred vasopressin antagonist is Tolvaptan (Otsuka America Pharmaceuticals). A preferred chemical for use as an antagonist against oxytocin and/or vasopressin is Atosiban (Ferring Pharmaceuticals, Sweden). Preferably, this lowers G-protein expression, such that children with ASD regain normal levels of Gαs and/or Gαq. In yet another preferred embodiment, the levels of G-proteins are altered by up-regulating expression of oxytocin and/or vasopressin. Preferably, up-regulation is achieved through the use of an oxytocin and/or vasopressin agonist. Preferably, this alters oxytocin and/or vasopressin levels, such that children with ASD regain normal levels of Gαs and/or Gαq. Once these normal G-protein levels are achieved, traditional therapy for autism can be administered, leading to a better response to treatment for the patient.

In a preferred embodiment, exposing PMBCs to vasopressin down-regulates G-protein expression, preferably Gαs and/or Gαq, in patients with ASD. Preferably, protein expression is down-regulated such that after exposure to vasopressin, patients with ASD have a level of G-protein expression that is less or lower than those patients without ASD. In preferred forms, G-protein expression is at least 10% less, even more preferably, at least 15% less, even more preferably, at least 20% less, even more preferably, at least 25% less, even more preferably, at least 28% less, still more preferably, at least 30% less, even more preferably, at least 37% less, still more preferably, at least 40% less, and, most preferably, at least 50% less. In a preferred embodiment, exposure to vasopressin results in down-regulation of Gαs, such that protein expression levels of Gαs, in patients with ASD, are about 37% lower than protein expression levels of Gαs in those patients without ASD. In another preferred embodiment, exposure to vasopressin results in down-regulation of Gαq, such that protein expression levels of Gαq, in patients with ASD, are about 28% less than protein expression levels of Gαq in those patients without ASD.

In another aspect of the present invention, a method for lowering scores on the Repetitive Behavior Scale is provided wherein such scores are lowered when oxytocin levels are altered. These oxytocin (OT) levels can be altered by the administration of OT agonists or up-regulators of OT expression, or antagonists or down-regulators of OT expression. Such administration of OT, an OT agonist, or an OT antagonist, can also be used for decreasing repetitive behavior compulsive behavior and/or ritualistic behavior in patients with ASD. It was found that lower OT levels were associated with higher rates of stereotyped behavior for those patients with ASD. This negative association was found both in boys and girls with ASD. The correlation found for the Autism Diagnostic Observations Schedule (ADOS) Stereotypy were 0.08 for the entire group of ASD patients and the correlation found for Autism Diagnostic Interview (ADI) Stereotypy was 0.12 for the entire group of ASD patients.

In another aspect of the present invention, a method for lowering scores on the Repetitive Behavior Scale is provided wherein such scores are lowered when vasopressin or OT levels are altered. These vasopressin or OT levels can be altered by the administration of vasopressin agonists or up-regulators of vasopressin expression; or antagonists or down-regulators of vasopressin expression. Such administration of vasopressin, a vasopressin agonist or a vasopressin antagonist can also be used for decreasing compulsive behavior and/or ritualistic behavior in patients with ASD.

In another aspect of the present invention, expression of G-proteins is modified in order to effect OT and/or vasopressin action in patients with ASD. Preferably, the OT action and/or vasopressin action lead to a diminished level of ASD characteristics. “Characteristics” and “Symptoms” of ASD, for purposes of the present invention, refer to any known or medically-accepted behavior, skill, or impairment associated with Autism Spectrum Disorders. Specific symptoms and characteristics include, but are not limited to, the following: impairment in social interaction, impairment in social development, impairment with communication, behavior problems, repetitive behavior, stereotypy, compulsive behavior, sameness, ritualistic behavior, restricted behavior, self-injury, unusual response to sensory stimuli, impairment in emotion, problems with emotional attachment, impaired communication, and combinations thereof.

In yet another aspect of the present invention, the measurement of Gαs and/or Gαq is used to monitor medication effects for Oxytocin, Oxytocin agonists, and/or Oxytocin antagonists, vasopressin, vasopressin agonists, and/or vasopressin antagonists, SSRI medication(s), and possibly for other medications as well.

Another aspect of the present invention provides for a method of managing ASD symptoms by monitoring and altering levels of G-proteins, preferably Gαs and Gαq, in children with ASD. In another aspect, treatment responses will be predicted by G-protein response. Boys with autism also appear to have altered OT levels. The cause for this is unknown, but might be important for helping to subgroup children with ASD.

In another embodiment of the present invention provides for a gene marker. This gene marker can be used as an indicator of autism. Preferably the gene markers are Gαs and Gαq. These gene markers can be utilized in order to determine a predisposition towards autistic behavior, as well as markers for other neurological disorders.

In yet another embodiment of the present invention, levels of Gαs and/or Gαq are used as a factor or factors in part of any medically-accepted screening and/or diagnostic method for ASD. Some preferred tests for ASD include, but are not limited to: Modified Checklist for Autism in Toddlers (M-CHAT), the Early Screening of Autistic Traits Questionnaire, and the First Year Inventory; the M-CHAT and its predecessor CHAT on children aged 18-30 months, Autism Diagnostic Interview (ADI), Autism Diagnostic Interview-Revised (ADI-R), the Autism Diagnostic Observation Schedule (ADOS) The Childhood Autism Rating Scale (CARS), and combinations thereof. The diagnostic factor of the present invention can be used to diagnose autism, where diagnosis includes the DSM-IV-TR, defined as an individual exhibiting at least six symptoms total, including at least two symptoms of qualitative impairment in social interaction, at least one symptom of qualitative impairment in communication, and at least one symptom of restricted and repetitive behavior. The diagnostic of the present invention adds an area of analysis to these established tests and comprises a screening of a patient's G-protein levels to determine if Gαs and/or Gαq were elevated, compared to G-protein levels in patients without ASD, and if the patient's levels are elevated this would be indicative of ASD. Preferably, the severity indicated by levels of G-proteins would increase as the G-protein levels increase.

A diagnostic test is also provided by the present invention. The diagnostic test comprises analysis of G-protein levels as an indicator for Autism. Additionally, this diagnostic is preferably used to determine indications of ASD in infants and young children as young as a few weeks old. Most of the traditional Autism diagnostics cannot be administered until a child reaches a certain level of cognition, usually around the age of 3 years. The diagnostic aspects of the present invention indicate the potential for ASD diagnosis, indication, or monitoring at a much earlier age. Elevated G-protein levels are used as an indicator that the patient should be screened and watched for symptoms of ASD. The younger a patient can be diagnosed, the earlier intervention can begin, including therapy and treatment, thereby giving the child a better long-term prognosis. In a preferred embodiment for young children or children for whom cognitive tests are a problem or challenge, the diagnostic is run on a patient at about 30 months of age or less, more preferably, at about 26 months of age, preferably, about 24 months of age, more preferably, at about 20 months of age, more preferably, about 18 months of age, even more preferably, at about 16 months of age, more preferably, at about 14 months of age, more preferably, at about 12 months of age, even more preferably, at about 11 months of age, even more preferably, at about 10 months of age, even more preferably, at about 9 months of age, more preferably, at about 8 months of age, even more preferably at about 7 months of age, more preferably, at about 6 months of age, more preferably, at about 5 months of age, even more preferably, at about 4 months of age, even more preferably, at about 3 months of age, even more preferably, at about 2 months of age, more preferably, at about 1 month of age, and even more preferably at or within a few days of birth.

For use in any diagnostic of the present invention, it is preferred that such a diagnosis be given to patients with ASD have Gαs and/or Gαq protein expression that is at least 15% greater than in patients without ASD, more preferably, at least 20% greater, even more preferably, at least 30% greater, even more preferably, at least 33% greater, still more preferably, at least 40% greater, even more preferably, at least 50% greater, even more preferably, at least 60% greater, even more preferably, at least 70% greater, more preferably, at least 80% greater, even more preferably at least 90% greater, still more preferably, at least 91% greater, and most preferably at least 100% greater. In a preferred embodiment, the expression of Gαs is at least 91% greater in patients with ASD, as compared with those patients who do not have ASD. In another preferred embodiment, the expression of Gαq is at least 33% greater, as compared with those patients who do not have ASD.

Because of the 4:1 ration of boys to girls with ASD, girls have been under-represented in studies of autism. It appears there are important sex differences in both OT and G-proteins which need to be considered in future studies.

For purposes of the present invention “patients with ASD” refers to those patients who have been or would be diagnosed with ASD according to known diagnostic screenings, including, but not limited to: Modified Checklist for Autism in Toddlers (M-CHAT), the Early Screening of Autistic Traits Questionnaire, and the First Year Inventory; the M-CHAT and its predecessor CHAT on children aged 18-30 months, Autism Diagnostic Interview (ADI), Autism Diagnostic Interview-Revised (ADI-R), the Autism Diagnostic Observation Schedule (ADOS) The Childhood Autism Rating Scale (CARS), and combinations thereof; or patients who exhibit any of the known symptoms, impairments, or behaviors associated with ASD, including, but not limited to: impairment in social interaction, impairment in social development, impairment with communication, behavior problems, repetitive behavior, stereotypy, compulsive behavior, sameness, ritualistic behavior, restricted behavior, self-injury, unusual response to sensory stimuli, impairment in emotion, problems with emotional attachment, impaired communication, and combinations thereof.

Patients “without ASD” or “not exhibiting symptoms” of ASD, refer to those patients that do not meet the criteria of “patients with ASD.”

### DESCRIPTION OF THE FIGURES

FIG. 1 is a graph illustrating Gαs mRNA in untreated PBMCs in children with ASD and controls;

FIG. 2 is a graph illustrating plasma OT levels in children with ASD and controls;

FIG. 3 is a graph illustrating Developmental Quotient Scores by Group and Gender;

FIG. 4 is a graph illustrating Gαq mRNA levels in the presence of vasopressin for patients with ASD compared to patients without ASD;

FIG. 5 is a graph illustrating Gαs mRNA levels in the presence of vasopressin for patients with ASD compared to patients without ASD;

FIG. 6 is a graph illustrating Gαq protein expression levels in the presence of vasopressin for patients with ASD compared to patients without ASD; and

FIG. 7 is a graph illustrating Gαs protein expression levels in the presence of vasopressin for patients with ASD compared to patients without ASD.

### DETAILED DESCRIPTION

The following examples describe representative embodiments of the present invention. It is understood that these examples are provided for representative purposes only and nothing herein shall be deemed a limitation on the overall scope of the invention.

### Example 1

This example determined whether autistic children displayed abnormalities in G-protein expression and function and whether expression and function of stimulatory G-proteins in the peripheral blood mononuclear cells (PMBCs) in females was greater than in males.

### Materials and Methods

Twelve (12) boys and twelve (12) girls with autism spectrum disorder (ASD), as well as 24 typically developing children (12 boys, 12 girls) to be used as controls were selected. All children were ages 4 or 5 years.

All children diagnosed with ASD were required to have either Autistic Disorder or PDD-NOS as determined by cut-off criteria on the Autism Diagnostic Observation Schedule—Generic (ADOS) and Autism Diagnostic Interview (ADI), and were also required to meet Diagnostic Statistical Manual, Fourth Edition (DSM-IV-TR) criteria as judged by 3 members of an experienced autism team. Ten boys had Autistic Disorder and 2 had PDD-NOS. Eleven girls had Autistic Disorder and 1 had PDD-NOS. Children who had known syndromes or definite dysmorphology were excluded, as were children on antiepileptic or psychotropic medications. Children taking medications that might impact immune function were excluded if they had taken such medication within 6 months of the visit.

Inclusion criteria for the children recruited as typically developing controls included: normal function in a regular preschool or kindergarten, T scores less than 70 in all domains on the Behavioral Assessment System for Children—Second Edition (BASC-2), no evidence of immune activation on physical exam, no psychotropic, antiepileptic or asthma/allergy medication within the last six months, and no first degree relative with ASD.

The mean age of the children with ASD was not significantly different than that of the controls (ASD=4.57 (0.54) and Controls=4.7 (0.61)). Twenty-one of the children with ASD were Caucasian, 2 were Hispanic, and 1 was African-American. Nineteen of the controls were Caucasian, 3 were Hispanic, and 2 were African-American. Although there was no difference between the groups in maternal education level, if classified as some college and no college, mothers of control children were significantly more likely to have graduate degrees (p=0.008) than mothers of children with autism.

Children with asthma and/or allergies were included so as long as they were not on medications. Because some of the children with autism were unable to complete the Kaufman Assessment Battery for Children (KABC-2) due to limited understanding of concepts necessary to complete the assessment, the Bayley Scales of Infant and Toddler Development—Third Edition (Bayley-III) was administered and a developmental quotient (DQ) calculated instead of excluding them. DQ was calculated by taking the child's mental age, as determined by the Bayley, divided by the child's chronological age in months, then multiplying that number by 100. “DQ” as used in this report includes the KABC-2 Mental Processing Index (16) or the calculated DQ from the Bayley (8).

TABLE 1
Eligibility for children in the study

| | |
|---|---|
| | Typically |
| Children | Developing |
| with Autism | Controls |

| Group | Girls | Boys | Girls | Boys | Total |
|---|---|---|---|---|---|
| Visit for eligibility | 15 | 20 | 13 | 14 | 62 |
| Failed eligibility | 2 | 5 | 1 | 1 | 9 |
| Failed blood draw | 1 | 3 | 0 | 1 | 5 |
| Testing complete | 12 | 12 | 12 | 12 | 48 |

C. Laboratory measures: There were some changes to the proposed laboratory studies as follows:
1. The oxytocin ELISA was elected rather than using sample for serotonin.
2. In addition to Gαq, Gαs was also studied for the following reasons:
a. G-proteins are redundant and G-coupled receptors promiscuous
b. Gαs hyperfunction has been reported in neuropsychiatric disease
c. Previous findings in this lab had found sex differences in Gαs in murine spleen cells as well as induction of Gαs expression in both sexes with exposure to OT,
d. Gαs is expressed in a much higher copy number compared to Gαq.
e. There are known disease states associated with both activating and inactivating mutations of the gene that codes for Gαs.

### Results

Both Gαs and Gαq mRNA were measured after PBMCs were incubated with vehicle (untreated condition) and after co-culture with hormone. For some subjects, the PBMC quantity yielded insufficient mRNA for PCR analysis. Gαs PCR was completed on 10 ASD girls, 9 control girls, 10 ASD boys, and 9 control boys. Gαq PCR was completed on 10 ASD girls, 8 control girls, 10 ASD boys, and 9 control boys.

There were differences in mRNA expression of both G-proteins (Gαq and Gαs) between children with ASD and controls.
1. Untreated PBMCs from children with ASD exhibited significantly higher Gαs expression (Mann Whitney U=101.00, p=0.021) compared to PBMCs from controls, with ASD children having a mean rank of 23.45. The mean rank for control children was 15.11. Upon PBMC exposure to OT the significant differences were no longer observed.
2. Males with ASD exhibited significantly increased Gαs mRNA expression compared with control males (Mann Whitney U=14.00, p=0.01) however there were not significant differences in Gαs mRNA expression in ASD females compared to controls (p=0.095). The ASD males had a mean rank of 13.10 whereas the control males had a mean rank of 6.56.
3. There was also significantly higher Gαq mRNA expression in untreated PBMCs from children with ASD compared to PBMCs from controls (Mann Whitney U=99.00, p=0.017). Upon exposure of these cells to OT these differences were no longer significant.
   There were also sex differences in expression of both stimulatory G-proteins:
4. Untreated PBMCs from all girls (ASD and controls) exhibited significantly higher

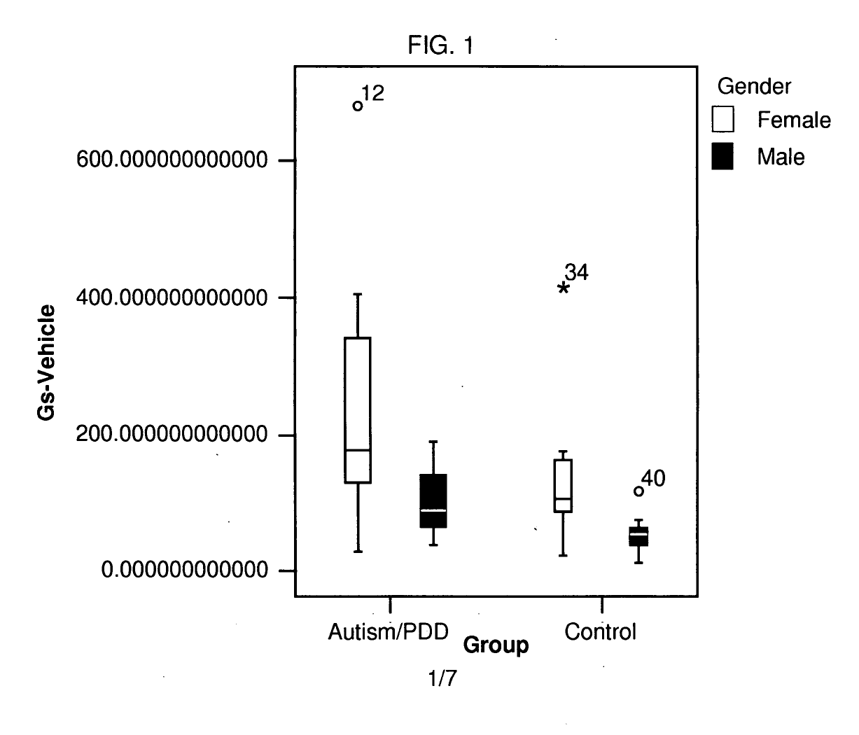

Gαs expression compared to those from all boys (U=79.00, p=0.002.) Mean rank was 24.84 for females and 14.16 for males. These differences were present in both ASD (p=0.029) and control (p=0.014) groups with females consistently having higher mean ranks (FIG. 1). These sex differences were not augmented with OT exposure in any group.
5. There was a trend toward increased Gαq mRNA expression in girls compared to boys in untreated PBMCs, which did not reach statistical significance. These differences were also not augmented with OT.

FIG. 1 illustrates Gαs mRNA in untreated PBMCs in children with ASD and controls. Expression of Gαq and Gαs mRNA were highly correlated in both untreated and OT treated PBMCs (Table 2). The Spearman correlation for the total sample was 0.64 for untreated and 0.71 for the OT treated conditions. Correlations of similar magnitude were found when we grouped the children according to gender and disease state. The notable exception was the relationship between Gαq and Gαs mRNA in untreated PBMCs from ASD males (Spearman correlation=0.04).

TABLE 2
Spearman Correlations between Gαq and Gαs in
untreated and OT treated PBMCs

| | Correlations | |
|---|---|---|
| | Gαq and Gαs (vehicle) | Gαq and Gαs (OT treated) |
| Total sample | .64** | .71** |
| ASD males | .04 | .52 |
| Control males | .87** | .87** |
| ASD females | .73* | .90** |
| Control females | .62 | .60 |

*P-value is <.05,
**P-value is <.01

Correlations between Gαq mRNA and plasma oxytocin (OT) and Gαs mRNA and plasma OT were also examined in untreated PBMCs (Table 3). The relationship between the G-proteins and plasma OT levels was generally not very strong. There was a strong relationship between Gαs mRNA and plasma OT in boys (Spearman correlation=0.57, p=0.01) This relationship appears to be due to strong correlations in the control group boys (Spearman correlation=0.57, p=0.11). Interestingly correlations between G-proteins and plasma OT were positive in controls and negative in children with ASD.

TABLE 3
Spearman Correlations between G-proteins in
untreated PBMCs and plasma OT levels

Correlations

| | Gαq and plasma OT | Gαs and plasma OT |
|---|---|---|
| ASD group | −.32 | −.31 |
| Control group | .34 | .57* |
| ASD males | −.26 | −.01 |
| Control males | .43 | .57 |
| ASD females | −.11 | −.19 |
| Control females | .32 | .28 |

*P-value is <.05,
**P-value is <.01

In untreated PBMCs, there were also correlations between G-protein mRNA and developmental and behavioral characteristics as shown in Table 4, and repetitive behaviors as shown in Table 5.

In the total sample of subjects, there was a significant negative relationship between Gαq mRNA and developmental quotient or DQ (Spearman correlation=−0.35, p<0.05). This negative correlation was also found between Gαs mRNA and DQ (Spearman correlation=−0.34, p<0.05). On the KABC-2, there was a significant negative correlation in the total sample between Gαq mRNA and scores on the Face Recognition (FR) subtest (Spearman correlation=−0.40, p<0.05) and between Gαs mRNA and FR (Spearman correlation=−0.47, p<0.05).

There was also a significant negative correlation between Gαs and the Vineland Social-Emotional Early Childhood Scales (SEEC) (Spearman=−0.50, p<0.05). This appears to be mostly attributable to ASD males as their correlation is also moderate and negative, while the correlation for control males was positive.

TABLE 4
Spearman Correlations between stimulatory G-proteins
and Developmental and Behavioral Characteristics

| Correlations | |
|---|---|
| Gαq | Gαs |

| | DQ | KABC-FR | DQ | KABC-FR |
|---|---|---|---|---|
| Total group | −.35* | −.40* | −.34* | −.47* |
| ASD group | −.24 | −.08 | −.10 | −.28 |
| Control group | .18 | −.28 | −11 | −.40 |
| ASD males | −.13 | −.01 | .10 | −.07 |
| Control males | .16 | .17 | −.08 | −04 |
| ASD females | −.16 | −.09 | .27 | −.11 |
| Control females | .39 | −.56 | −.36 | −.50 |

P-value is <.05,
**P-value is <.01

TABLE 5
Spearman Correlations between G proteins and
Repetitive Behavior Scale Scores

| | Correlations | | | |
|---|---|---|---|---|
| | Gαq | | Gαs | |
| | ASD girls | ASD boys | ASD girls | ASD boys |
| Stereotyped behavior-SB | −.13 | .19 | −.25 | .41 |
| Self-injurious behavior- | −.08 | −.65* | −.26 | .27 |
| SIB | | | | |
| Compulsive behavior-CB | −.48 | .14 | −.63* | .40 |
| Ritualistic behavior-RB | −.49 | .26 | −.26 | .25 |
| Sameness behavior-Sa B | −.64* | −.23 | −.62* | .35 |
| Restricted behavior-RSB | −.40 | .27 | −.36 | .46 |
| Total | −.38 | .03 | −.52 | .47 |

P-value is <.05,
**P-value is <.01

For the total sample of ASD children, there was not a significant relationship between Repetitive Behavior Scales (RBS) scores and G-proteins. However, within gender groups there were a few significant relationships. For girls with ASD, there was a significant negative relationship between Gαq mRNA and the Sameness behavior subscale score (Spearman=−0.64, p<0.05). There was also a significant negative relationship (Spearman=−0.62) between Sameness behavior scores and Gαs mRNA for females. On the Compulsive behavior subscale, scores were negatively related to Gαs mRNA in females with ASD (Spearman=−0.63). For boys with ASD, the only significant correlation was on the Self-Injurious Behavior subscale (Spearman=−0.65, p<0.05).

### Example 2

This example determined whether prepubertal children with autism exhibited altered levels of OT compared to unaffected sex-matched controls. This example also determined if there were sex differences in OT levels and if OT levels correlated with measures of social impairment or motor stereotypy.

### Materials and Methods

All 48 children had blood drawn for plasma oxytocin (OT) levels. Determination of OT was performed using a 96-well plate commercial OT enzyme-linked immunosorbent assay (ELISA) kit. Samples were run in duplicate and repeated in two separate experiments when plasma volume was sufficient (42/48).

Based on the paper by Modahl et al (1998)', it was hypothesized that OT levels would be lower in children with ASD as compared to controls and that OT levels would correlate with deficits in social impairment (social recognition) and motor stereotypy. It was also proposed that there might be sex differences in OT levels.

Plasma OT levels in children with ASD as compared to typically developing controls:
1. This study did not find differences in plasma OT levels between children with ASD and controls when data from both genders were combined (Mann Whitney U=34.50, p=0.27). The ASD children exhibited a trend toward higher plasma OT levels (Mean rank=26.73) compared to control children (Mean rank of 22.27).
2. For male children there were significant differences between the control group and the ASD group in plasma OT levels (Mann Whitney U=30.50, p=0.01). The differences were opposite of the Modahl report as the ASD group exhibited higher mean levels (Mean rank 15.96) compared to the control group (Mean rank 9.04). ASD females (Mean rank 11.17) exhibited a trend toward lower levels of oxytocin compared to control group (Mean rank 13.83) however, this did not reach statistical significance (Mann Whitney U=56.00, p=0.36).
   Plasma OT levels in boys versus girls:
3. ASD boys exhibited significantly higher OT levels (Mean rank=15.67) compared to ASD girls (Mean Rank=9.33; Mann Whitney U=34.00, p=0.028).
4. There were no significant differences in OT levels between control girls and control boys (U=47.00, p=0.16) although there was a trend for control girls (Mean rank=14.58) to have higher OT levels than boys (Mean rank=10.42).

TABLE 6
Levels of OT in children with ASD and
typically developing controls

| | ASD boys | Control boys | ASD girls | Control girls |
|---|---|---|---|---|
| μ (SD) | 16.26 (2.48) | 12.85 (3.21) | 13.13 (3.15) | 14.66 (3.09) |
| Range | 13.74-22.99 | 7.51-18.26 | 7.24-17.59 | 10.72-21.46 |

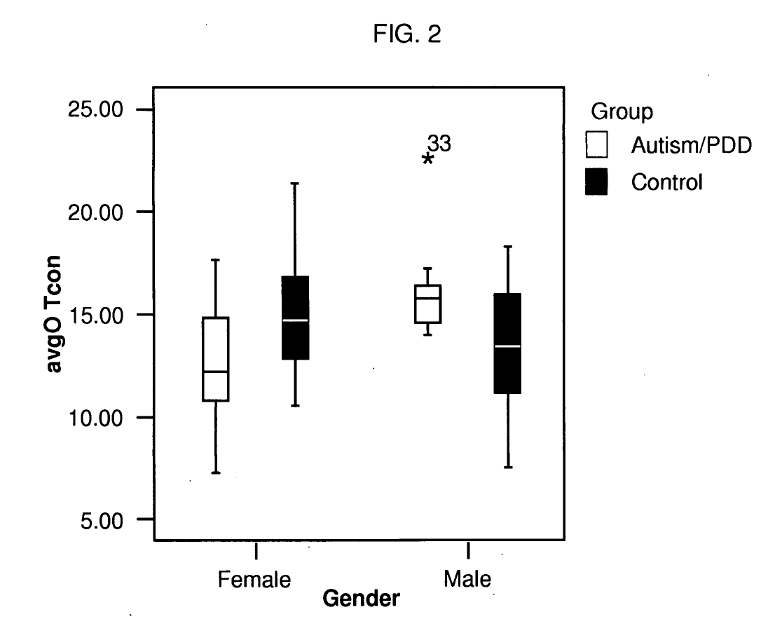

Similar to the findings of Modahl et al., there was significant overlap in plasma OT levels. FIG. 2 illustrates plasma OT levels in children with ASD and controls.

Also similar to the findings by Modahl, correlations between plasma OT levels and some developmental and behavioral measures were found (Table 6). Scores (Likert scale) measuring emotional reaction to the blood draw were not related to plasma OT levels in any group.

For males there is a significant negative correlation between OT and the Vineland SEEC scores for boys in the total sample. Higher levels of OT are related to lower social skills. This association was strongest for control boys.

There was a significant negative correlation for control males between OT levels and scores on the Hand Movement (HM) subscale of the KABC-2.

For ASD females and control males there was a significant negative correlation between plasma OT and DQ.

For ASD males, DQ was highly correlated with the FR subtest of the KABC-2. While ASD females had a similarly strong association between DQ and scores on the HM subscale of the KABC-2, ASD females had little correlation between DQ and FR. The correlations between DQ, FR, and HM for control children were moderate but nonsignificant in this sample.

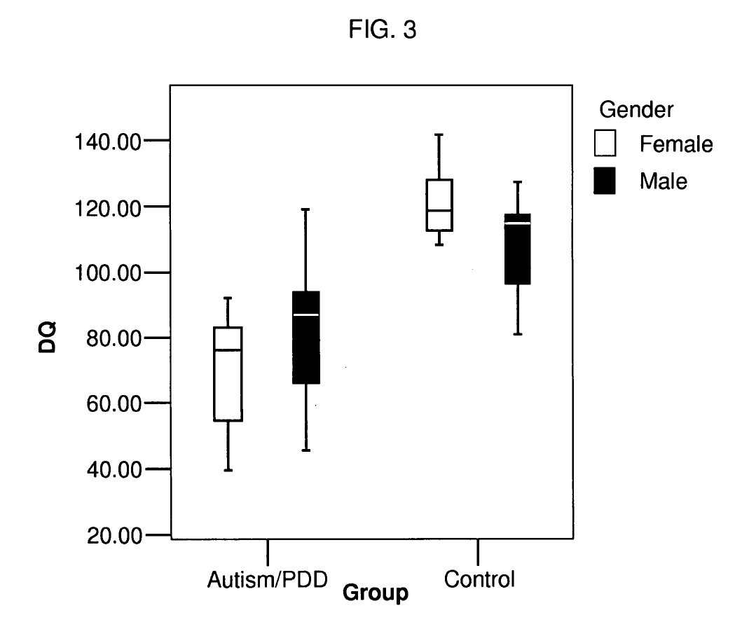

Control group girls (Mean rank 15.42) exhibited significantly higher (Mann Whitney U=34.00, p=0.028) DQ than control group boys (Mean rank 9.58). For the ASD group the difference in DQ between boys (Mean rank 14.96) and girls (Mean rank 10.04) was not statistically significant, but the p value was relatively small (Mann Whitney U=42.5, p=0.09). These differences can be seen in FIG. 3.

TABLE 7
Correlations between plasma OT and
Developmental and Social Measures

Correlations

| | OT and | OT and | OT and | OT and |
|---|---|---|---|---|
| | DQ | FR | HM | SEEC |
| Total sample | −.19 | −.10 | −.27 | −.19 |
| ASD group | −.14 | −.09 | −.01 | −.15 |
| Control group | −.19 | .09 | −.46* | −.06 |
| Males (all) | −.43* | −.28 | −.48* | −.56* |
| Females (all) | −.05 | .04 | −.08 | .10 |
| ASD males | −.08 | .02 | 0 | −.27 |
| ASD females | −.59* | −.41 | −.03 | −.38 |
| Control males | −.61* | .10 | −.63* | −.53 |
| Control females | −.21 | .14 | −.37 | .13 |

*p-value is <.05;
**p-value is <.01

Because of the significant (or small p-value) gender differences in DQ within groups, DQ was used as a covariate in within-group analyses examining the effect of gender on cognitively related outcomes, to ensure that findings (or lack of findings) could not be attributable to differences in DQ. Analysis of Covariance was used with DQ as the covariate and gender as the effect of interest. When examining the difference in OT levels based on gender within groups, the effects were actually larger for both ASD and control children when controlling for DQ levels. The effect remained significant for ASD children with males having significantly higher OT levels. For the control group, the p-value for gender was relatively small (0.10) and the partial eta squared (0.13) suggested a moderate effect with females having higher OT levels than males.

When the developmental quotient was controlled, the negative correlation between OT and HM scores was still significant and negative. Likewise, when the DQ was controlled, the negative relationship between SEEC and OT for males was reduced slightly but remained strong. When controlling for DQ and examining the relationship between OT and HM in males, however, the correlation was reduced by about 40% and was no longer a statistically significant correlation. FIG. 3 illustrates Developmental Quotient Scores by Group and Gender.

Correlations between plasma OT and social and communication scores on the ADOS and the ADI were also examined (Table 8). None of the correlations reached statistical significance.

TABLE 8
Correlations: Plasma OT and Social/Communication Scores
on the ADOS and ADI

| | Correlations | | |
|---|---|---|---|
| | ADOS Social/ | | ADI |
| | Communication | ADI Social | Communication |
| | n = 17 | n = 24 | n = 24 |
| ASD group | .26 | .13 | .10 |
| ASD males | .41 | .26 | .42 |
| ASD females | .17 | .17 | .29 |

*p-value is <.05;
**p-value is <.01

When the DQ was controlled, the relationship between ADOS Social/Communication and OT level actually increased in the total sample. For females the relationship was reduced from a Pearson correlation of 0.46 to 0.31. For males, the correlation between OT and ADOS Social/Communication was not changed by controlling for DQ.

The relationship between repetitive behaviors and plasma OT levels using scores from both the ADOS and the ADI was also studied (Table 9). There were no significant correlations. The relationship between OT and ADOS stereotypy was strengthened by controlling for DQ in the total sample. The correlation within males was not affected and the correlation for females was near zero when controlling for DQ.

TABLE 9
Correlations between Plasma OT and Stereotypy Scores
on the ADOS and ADI

Correlations

| | ADOS Stereotypy | ADI Stereotypy |
|---|---|---|
| | n = 17 | n = 24 |
| ASD group | .08 | .12 |
| ASD males | .30 | .18 |
| ASD females | −.16 | .36 |

Significant correlations were found on the Repetitive Behavior Scale (RBS), with lower OT levels associated with higher rates of stereotyped behavior for the ASD group as a whole (Spearman=−0.47, p<0.05). This negative association was present in both the boys and girls with ASD. For the boys there were also significant negative correlations between OT levels and compulsive behavior, ritualistic behavior, and total RBS score. Correlations between OT and sameness behavior and restricted behavior for boys were also quite large and negative, although not statistically significant (Table 10).

TABLE 10
Correlations between Plasma OT and Scores on the Repetitive Behavior Scale

| | | | | | | | RBS |
|---|---|---|---|---|---|---|---|
| Correlations | RBS:SB | RBS:SIB | RBS:CB | RBS:RB | RBS:Sa | RBS:RES | (total) |
| ASD group | −.47* | .02 | −.29 | −.28 | −.14 | −.28 | −.27 |
| ASD males | −.54 | .06 | −.61* | −.68* | −.44 | −.52 | −.62* |
| ASD females | −.36 | .29 | −.09 | −.37 | −.04 | −.23 | −.11 |

*p-value is <.05;
**p-value is <.01

Correlations between OT levels and the Behavioral Development Questionnaire (BDQ, adapted from the Wing) scores were also examined. None of the correlations between plasma OT levels and BDQ subtypes (aloof, passive, and active but odd) were statistically significant.

### Example 3

This example determined whether there were differences in immune activation and B and T lymphocyte function in autistic children compared to controls, and if sex differences in responsiveness to OT correlate with sex differences in lymphocyte function and cytokine levels.

High levels of neopterin indicate monocyte/macrophage activation, as well as activation of T-cells and cell-mediated immunity. High blood monocyte counts and high neopterin levels were previously reported in a sample of autistic children². These studies were repeated. TNF-alpha and IL-6 levels, both of which have been reported to be increased in autism^{3,4} were also investigated. Eligibility criteria were fairly strict, and children with severe chronic medical problems and children on medications were not included. There were no significant differences between groups in numbers of children with allergy, chronic ear infection, or both compared to children with no medical problems (Fisher's exact test p=0.39). The findings were as follows:
1. There were no significant differences in neopterin levels between ASD and control children (Mann Whitney U=25.00, p=0.43). There were no significant gender differences across all the children (p=0.65) and no significant gender differences within control (p=0.67) and ASD (p=0.84) groups. NO differences in absolute monocyte counts between children with ASD and controls were found.
2. Neopterin and TNF-α in the supernatant of PBMCs treated with OT were significantly correlated for the total sample (Spearman rho=0.41, p<0.01). A strong relationship was found among these two variables for the control group and for females. This relationship was the strongest for control females in the study (Rho=0.79, p<0.01). For control females, neopterin was also significantly correlated with IL6 in both the oxytocin treated and vehicle conditions.
3. There were no significant differences between control and ASD groups in TNF-α in supernatant of PBMCs regardless of OT exposure. There were no significant gender differences for TNF-α within the ASD group or within the control group.
4. There were no significant differences between control and ASD groups in IL-6 in supernatant of PBMCs regardless of OT exposure. There were not significant gender differences for IL-6 within the ASD group or within the control group.
5. IL6 and Gαs were strongly correlated with each other for males in the current sample (Table 11). The direction of the correlation differs based on group. For ASD males there was a significant negative correlation (Spearman's rho=−0.70, P<0.05) and for control males the correlation was positive (rho=0.57, p=0.11). There was also a very large negative correlation between IL6 in supernatant of untreated PBMCs and Gαs mRNA for control females (rho=−0.80, p<0.01). There were no significant gender differences within the ASD group or within the control group.

TABLE 11
Spearman Correlations between G proteins and IL-6

Correlations

| | Gαs and IL-6 | Gαq and IL-6 |
|---|---|---|
| | (supernatant) | (supernatant) |
| Total sample | −.38* | −.18 |
| ASD group | −.43 | −.22 |
| Control group | .21 | −.06 |
| ASD males | −.64* | −.19 |
| Control males | .45 | .18 |
| ASD females | −.22 | .08 |
| Control females | .80** | −.07 |

*P-value is <.05,
**P-value is <.01

### Discussion:

### Increased G Proteins: Parallels to Neuropsychiatric Conditions:

Multiple neurotransmitter systems have been implicated in the pathogenesis of autism, including the serotonergic, dopaminergic, and glutaminergic systems.^{5,6} Oxytocin and vasopressin have also been implicated because of their role in social affiliation, which is impaired in persons with autism.⁷⁻⁹ Heterotrimeric G-proteins are critical in post-receptor information transduction.¹⁰⁻¹¹ Abnormalities in both G-proteins and in members of the G-protein signaling (RGS) family have been proposed to cause functional imbalance in multiple neurotransmitter pathways.¹² Neurotransmitter imbalance has been hypothesized to account for the diverse clinical features found in the neuropsychiatric disorders and in autism^{6,12}.

Dysregulated G-protein function in peripheral blood mononuclear leukocytes (PBMCs) is a well established finding in both bipolar disorder¹² and in schizophrenia.¹¹ It was found that untreated PBMCs from children with autism spectrum disorder (ASD) exhibited significantly higher Gαs and Gαq compared to PBMCs from controls. This is the first report of dysregulated stimulatory G-proteins in autism spectrum disorder.

There is accumulating evidence that antidepressant and antimanic drugs exert effects on signal transduction mechanisms, particularly via Gαs. In both bipolar illness and schizophrenia, Gαs mRNA synthesis in PBMCs can be decreased by drug treatments. Karege et al (2000)¹³ reported that lithium decreased both Gαs mRNA synthesis and protein levels in a group of 15 subjects with bipolar disorder, and Avissar¹⁴ et al showed that G-protein measurement PBMCs of patients with schizophrenia can be used to biochemically monitor the effects of antipsychotic medications.

The children in this study were all prepubertal and on no medications. Many children with ASD do benefit from psychotropic medications, including SSRI medications for anxiety and obsessive-compulsive behaviors and atypical neuroleptics for disruptive behaviors. SSRI medications have been shown to increase plasma oxytocin levels acutely.^{15,16} In this pilot work, it was found that PBMCs from children with ASD treated in vitro with OT did not have the significantly increased Gαs mRNA seen in PBMCs in the untreated (vehicle) condition. Based on the findings herein, the measurement of Gαs will be helpful in monitoring medication effects for SSRI medication, and possibly for other medications as well.

### Increased G Proteins: Possible Parallels to Glutamate Signaling in Fragile X Syndrome

Glutamate is the major excitatory neurotransmitter in the central nervous system. Glutamate signals both through ligand-gated ion channels and through G protein-coupled metabotropic receptors (mGluRs). Metabotropic glutamate receptor 5 (mGluR5) has recently been implicated in the pathogenesis of Fragile X syndrome,¹⁷ which shares many of the behaviors associated with autism, including increased risk for seizures, cognitive impairments, hypersensitivity to tactile stimuli, and social deficits. Approximately half of children with Fragile X have enough autistic behaviors to meet criteria for an ASD diagnosis. Although most children with ASD do not have the silencing of the FMR-1 gene that defines Fragile X syndrome, studies with Fmr1 (encodes the Fragile X mental retardation protein, FMRP) and Grm5 (encodes mGluR5) mutant mice have helped to define not only the role of FRMP but also of metabotropic glutamate receptor 5 in memory and learning.¹⁷ The mGlu5 receptor is important to long term depression (LTD) which is important in normal brain development so that unstimulated synaptic connections can be eliminated.¹⁸ There is evidence for abnormal synaptic pruning in ASD.¹⁹ In Fragile X syndrome, the absence of FRMP leads to enhanced LTD which results in weak and immature synaptic connections. G-proteins have been shown to act as “rheostats”²⁹ on metabotropic glutamate receptors, and it is possible that upregulation of G-proteins may cause abnormal signaling at the mGlu5 receptor.²¹ Indeed, Bourtchouladze et al²² showed that in a transgenic mouse model, chronically increased Gαs disrupted associative and spatial learning.

### G Proteins: Sex Differences

In this pilot work, there were significant sex differences in Gαs mRNA expression in children with ASD and in typically developing children. Thus, this study demonstrated that studies that explore G-proteins and neurotransmitters should be controlled for gender. It is interesting that sex differences in expression of G-proteins were demonstrated even in these young prepubertal children. Gonadal steroids (androgens and estrogens) are normally very sexually dimorphic during in utero development and during the first few weeks or months of life. After a few months of age, androgen and estrogen levels are low in both boys and girls until the onset of puberty. It is possible that gonadal steroid exposure earlier in life or even antenatally induces some lasting sexual dimorphism in G-protein expression.

### Oxytocin in Autism

Like Modahl¹ this study also found evidence for dysregulation of plasma OT in autism. Contrary to the findings of Modahl, boys with ASD had higher plasma OT levels as compared to male controls. This study did not find that OT levels in girls were significantly different than in typically developing girls, although ASD girls tended to have lower plasma OT levels. Motor stereotypy was negatively associated with plasma OT in the children with ASD. Finally, there were no significant differences in OT levels between control girls and control boys, but there were significant gender differences in OT in the children with ASD. Both behavioral and biological sex differences should be studied in children with ASD.

Some of the findings in this study were similar to those reported in Modahl's 1998 study of plasma OT in boys with ASD. Like Modahl it was found that OT levels were not correlated with DQ in either the ASD group or the control group. It was also found that the correlations between OT levels and behavioral variables in the ASD group to be in the negative direction, with the exception of small positive correlations between OT and SIB in the ASD group, and OT and FR in ASD males.

Both this study and Modahl's study suggest dysregulation of OT in ASD. However, in this study, higher mean levels in children with ASD were found as compared to controls instead of the lower mean plasma levels reported by Modahl. It is possible that this discrepancy may be due to differences in the samples herein. The subjects of this study were all four or five years of age. It is possible that ASD males may have higher plasma OT at ages four and five, but then their plasma OT levels do not increase with age as in typically developing children. Thus by ages six to ten (the ages of children in the Modahl study), the ASD males might have lower OT levels than control males. Plasma OT levels in females with ASD have not previously been studied.

It is also possible that medications may account for the differences in the findings in this study. This study excluded children who were on any medications. Ten of the 29 participants in the Modahl study were on medications, although when examined individually there were no outliers and so they were included in the overall analysis. Because some medications (e.g. the selective serotonin reuptake inhibitors) can increase plasma OT, future studies of OT in ASD should control for medications.

Finally, the measurements of plasma OT in this study were via ELISA rather than radioimmunoassay (RIA) as done by the Modahl study since there were no radioisotopes in this lab for RIA. Although both are considered reliable,²³ it is possible that there may be some differences in results.

### Oxytocin in Autism: Relationship to Serotonin

In addition to dysregulation of OT, more than 25% of children with ASD have high serotonin levels.²⁴ It is unclear if elevated hormone levels can result in upregulation of G-protein signal transducers. It is also unknown whether children with high serotonin levels are also more likely to have high OT levels. Serotonin (5-HT) stimulation in the paraventricular nucleus of the hypothalamus (PVN) increases plasma oxytocin levels. This occurs via both 5-HT1A and 5-HT2A receptors. For the 5-HT2A receptors, this release is dependent on Gq signaling (i.e., 5-HT2A receptors couple to Gq) so increases in Gq in the PVN would result in increased release of oxytocin into the plasma.

### Oxytocin in Autism: Intervention Possibilities

Oxytocin is thought to be important to social interaction and to repetitive behavior. Hollander²⁵ reported that OT infusion in adults with ASD improved affective speech comprehension, a measure of auditory processing of social stimuli. Hollander²⁶ also studied the effect of intravenous OT on repetitive behaviors in adults with ASD. He reported that the severity and number of different types of repetitive behaviors decreased with OT infusion. OT has also been given intra-nasally to male university students to study its role in facilitating and mediating social interactions^{28,29} and the results revealed that OT might increase trust and prosocial behavior.

This study examined social impairment using the KABC-FR, KABC-HM, SEEC, ADOS and ADI social/communication variables. The sample size was small, and the results herein did not reach statistical significance, however a negative correlation between OT and Vineland Social Emotional Early Childhood Scale (SEEC) in both ASD males and ASD females (Spearman's correlation −0.27 and −0.38, respectively) was found. A positive correlation between plasma OT levels and the ADOS and ADI scores was also found, and as OT levels increased, the ADOS and ADI scores increased indicating more social/communication problems. These findings are consistent with Modahl's findings. Modahl also found a significant negative correlation in their ASD group with OT levels and socialization items. A relationship between OT and a variety of repetitive behaviors on the RBS, with higher plasma OT levels correlating with fewer repetitive behaviors was also found.

The relationship between baseline plasma OT and response to OT infusion should be studied in persons with ASD to see if plasma OT is a biomarker for response to OT or to other medications that can increase OT (such as the SSRI medications).

### Example 4

This example is provided to illustrate the differences between the amount of mRNA for the G-proteins Gαs and Gαq when cells are exposed to vasopressin.

### Materials and Methods

Samples were taken from the patients, as described in Example 1, and mRNA was isolated from cells exposed to vasopressin and analyzed for amount of mRNA expressing Gαs and Gαq.

Venipuncture. 3 ml/kg of blood (to a maximum of 40 cc) were drawn after a 2 hour fast between 10 am and 12 am as previously described (Modahl). EMLA (topical lidocaine cream) was placed on the antecubital region of both arms and covered with tegaderm prior to blood draw. 10 ml of blood was collected in heparinized tubes on ice until processing within 15 minutes. This aliquot was centrifuged at 4° C. at 3000 g for 20 minutes, after which the plasma was stored in 1 ml aliquots at −80° C. until further processing, at which time it was transported on dry ice to the laboratory. The remaining blood (up to 30 cc) was placed on ice and transported within 30 minutes of blood draw to the same lab.
PBMC isolation, hormone treatment and cell culture. PBMCs were isolated from heparinized blood using Histopaque 1077 (Sigma-Aldrich, St. Louis, Mo.). Briefly, 5 ml of blood was diluted 1:1 in PBS and carefully layered onto 3 ml of Histopaque 1077 in a 50 ml conical centrifuge tube. Tubes were centrifuged at 400 g for 30 min at RT. The buffy coat was removed from the tube and washed three times in complete RPMI without phenol red. Cell counts and viability were determined using Trypan blue. Cell viability was >95%. Resultant PBMC's were suspended in complete RPMI with 10% charcoal stripped, dilapidated fetal calf serum (FCS) (Invitrogen, Carlsbad, Calif.) and added in 1 ml aliquots to 12 well plate with vehicle and vasopressin (10−8M) in RPMI+10% charcoal stripped, dilapidated FCS. Cells were incubated overnight (18-24 hr) before wells were harvested and rinsed. Cell suspensions were centrifuged at 500 g for 10 min at RT, and supernatant was aspirated. Buffer RLT Plus from the RNeasy Mini Plus Kit (QIAgen, Valencia, Calif.) was added to the cell pellets before vortexing to lyse cells. Samples were stored at −80° C. before RNA purification and isolation.

RNA isolation and quantitation. Samples were thawed, vortexed, and placed in QIAshredder columns. Columns were centrifuged for 2 min at 15000 g. The homogenized lysate was transferred to a gDNA Eliminator spin column and centrifuged for 30 sec at 9000 g. Equal volumes of 70% ethanol were added to the eluent. The mixture was transferred to RNeasy spin columns and centrifuged for 15 sec at 9000 g. Columns were washed with Buffer RW1 and the Buffer RPE, centrifuging for 15 sec at 9000 g, each time discarding the eluent. Buffer RPE was again added and columns centrifuged for 2 min at 9000 g to dry the column. Columns were then centrifuged for 1 min at 15000 g. RNA was eluted with the addition of Nuclease-free water and spun for 1 min at 9000 g. RNA was examined by spectrophotometry and quantitated using the Ribogreen assay (Invitrogen, Carlsbad, Calif.). Samples were diluted 1:200. Standards of 0, 20, 100, 500, and 1000 ng/ml were made from E. coli ribosomal RNA in TE buffer. Ribogreen reagent (1:200) was added to samples and standards, and samples were read on a Bio-Tek FLₓ800 fluorescent plate reader. RNA was diluted in nuclease-free water to a final concentration of 150 ng/ml cDNA.

cDNA construction and one-step real-time RT-PCR. Reverse transcription was performed using the SuperScript II Reverse Transcription Kit (Invitrogen, Carlsbad, Calif.). First, any residual DNA was digested away with DNase I for 15 min, with DNase activity subsequently quenched with 25 mM EDTA at 65° C. for 15 min. 10 mM dNTP and 0.5 ug/ml oligo (dT) were added to DNase-treated samples at 65° C. for 5 min. Samples were placed on ice, and reaction mixture containing DEPC-treated water, 25 mM MgCl₂, 10×RT buffer, 0.1 M dithiothreitol, and RNaseOUT recombinant RNase inhibitor was added at 42° C. After 2 min, SuperScript II Reverse transcriptase was added and incubated for 50 min at 42° C. Samples were cooled, and RNase H was added at 37° C. for 20 min. DEPC-treated water was added to make a final volume of 50 μl of 150 ng/ml cDNA.

One-step real-time RT-PCR was performed using the SYBR Green PCR kit (Bio-Rad, Hercules, Calif.). GAPDH and G protein sequences were obtained from the Gene Bank database and the following primers were constructed using the GenBank sequence # NM_{—}080426: Gα_{S} sense: 5′-TCT ACC GGG CCA CGC ACC GC-3′ (SEQ ID NO. 1); Gα_{S} antisense: 5′-GCA GGA TCC TCA TCT GCT TC-3′ (SEQ ID NO. 2); Gα_{Q} sense: 5′-GAT GTT CGT GGA CCT GAA CC-3′ (SEQ ID NO. 3); Gα_{Q} antisense: 5′-CAA CTG GAC GAT GGT GTC CT-3′ (SEQ ID NO. 4); GAPDH sense: 5′-TGA CAA CTT TGG TAT CGT GGA AGG-3′ (SEQ ID NO. 5); GAPDH antisense: 5′-AGG GAT GAT GTT CTG GAG AGC C-3′ (SEQ ID NO. 6). A BLAST search was performed using the National Center for Biotechnology Information's BLAST WWW Server. RT-PCR was performed on the iCycler (Bio-Rad, Hercules, Calif.). The following parameters were used for the RT-PCR program: 95° C. at 3 min; 35 cycles of 95° C. at 20 sec, 56° C. at 20 sec, 72° C. for 20 sec; 95° C. for 1 min; and 55° C. at 1 min. Data were expressed as the ratio of the gene of interest compared to a housekeeping gene (GAPDH). That ratio was expressed as a percent of control. Control cDNA was created from a pool of PBMC's from healthy adult males and females and was used throughout the study.

### Results and Conclusion

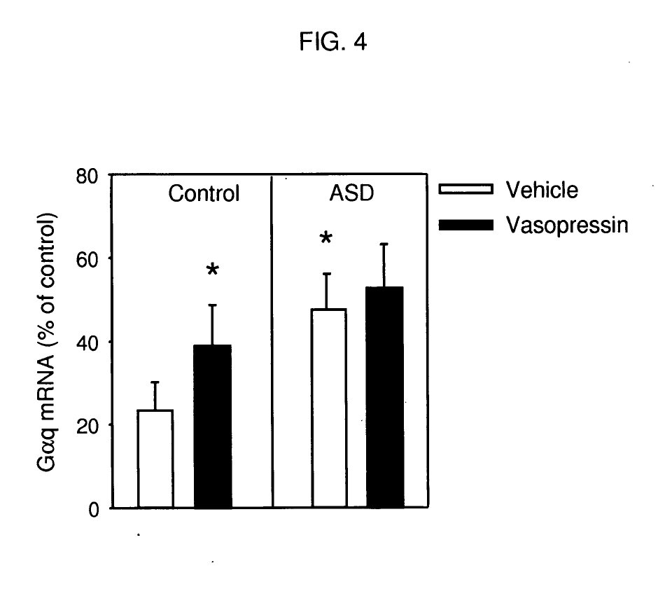

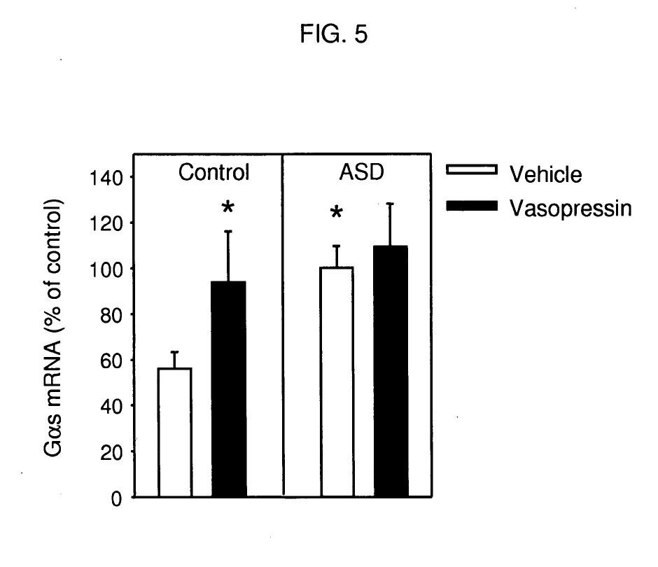

The results showed that children with ASD typically have higher G-protein mRNA levels than those children who do not exhibit any signs of ASD. This can be seen in FIGS. 4 and 5, when you compare G-protein mRNA with the control vehicle in the control group and the ASD groups. The difference in G-protein mRNA was found to be statistically significant. The statistically significant values are represented in FIG. 4, comparing levels of Gαq mRNA, and FIG. 5, comparing levels of Gαs mRNA. The results are also summarized in Table 12 and Table 13 below. This finding shows that patients with ASD have higher levels of G-protein mRNA, specifically Gαs mRNA and Gαq mRNA. The results also showed that cells exposed to vasopressin had increased Gαs mRNA and Gαq mRNA for healthy patients but not for patients with of ASD.

TABLE 12
Gαs mRNA after exposure to vasopressin
GαsmRNA

| | Control | Autism | Control | Autism |
|---|---|---|---|---|
| | Vehicle | Vehicle | Vasopressin | Vasopressin |
| Gαs mRNA | 22.6 | 123.1 | 33.1 | 78.5 |
| (% of control) | 53.7 | 75.9 | 66.1 | 43.7 |
| | 50.2 | 90.1 | 30.8 | 214.4 |
| | 127.2 | 55.4 | 200 | 59.5 |
| | 126.5 | 254.9 | 245.8 | 151.6 |
| | 48.5 | 151.3 | 137.6 | 373.2 |
| | 46.1 | 81.2 | 66.3 | 65.9 |
| | 45.1 | 165.9 | 42.7 | 181.8 |
| | 100 | 114.9 | 58.3 | 66.3 |
| | 28.7 | 85.9 | 120 | 52.6 |
| | 129.9 | 20.5 | 31.9 | 77 |
| | 63.1 | 50 | | 62 |
| | 51 | 144.1 | | 54 |
| | 121.8 | 92.3 | | 21.1 |
| | 40.9 | 59.2 | | 114.8 |
| | 28.5 | 88.3 | | 144.4 |
| | 33.15 | 67.5 | | 67.6 |
| | 25.9 | 60.3 | | 99.7 |
| | 17.8 | 71.8 | | 147.3 |
| | 11.1 | 42.1 | | |
| | 53.6 | 138.6 | | |
| | 24.1 | 199.9 | | |
| | 44.7 | 118.9 | | |
| | 47.9 | 81 | | |
| | | 113.9 | | |
| | | 26.1 | | |
| | | 24.1 | | |
| | | 33.4 | | |
| | | 83.6 | | |
| | | 77.4 | | |
| | | 127.7 | | |
| | | 166.4 | | |
| | | 101.9 | | |
| | | 216 | | |
| Average | 55.9 | 100.1 | 93.9 | 109.1 |

TABLE 13
Gαq mRNA after exposure to vasopressin
GαqmRNA

| | Control | Autism | Control | Autism |
|---|---|---|---|---|
| | Vehicle | Vehicle | Vasopressin | Vasopressin |
| Gαq mRNA | 16.5 | 22.29 | 16.42 | 8.62 |
| (% of control) | 132.2 | 9.11 | 24.87 | 1.94 |
| | 27.7 | 19.57 | 26.4 | 30.78 |
| | 78.5 | 11.69 | 9.17 | 7.45 |
| | 36.7 | 50.45 | 3.36 | 48.7 |
| | 4.48 | 152.94 | 54.5 | 96.6 |
| | 41.11 | 151.85 | 23.7 | 171.8 |
| | 37.33 | 16.85 | 1.04 | 43.6 |
| | 7.11 | 53.88 | 12.94 | 37.48 |
| | 4.64 | 7.6 | 1.11 | 13.87 |
| | 33.94 | 18.3 | 100.5 | 12.07 |
| | 8.2 | 4.1 | 59.8 | 24.1 |
| | 7.18 | 18.2 | 146.7 | 7.99 |
| | 6.74 | 48 | 45.1 | 48.3 |
| | 3.6 | 61.26 | | 90.1 |
| | 9.5 | 60 | | 96.6 |
| | 9.8 | 16.7 | | 90.18 |
| | 10.12 | 87.4 | | 107.8 |
| | 1.98 | 9.47 | | |
| | 9.75 | 3.7 | | |
| | 3.38 | 10.54 | | |
| | | 9.03 | | |
| | | 107.1 | | |
| | | 137.3 | | |
| | | 59.6 | | |
| | | 151.7 | | |
| | | 35.03 | | |
| | | 79.71 | | |
| | | 31.64 | | |
| | | 15.82 | | |
| | | 17.2 | | |
| Average | 23.4 | 47.7 | 37.5 | 52.11 |

### Example 5

This example illustrates the correlation between oxytocin exposure and mRNA amounts for G-proteins, specifically Gαs mRNA and Gαq mRNA.

### Materials and Methods

Samples were taken from the patients, as described in Example 1, and mRNA was isolated from cells exposed to oxytocin and analyzed for amount of mRNA expressing Gαs and Gαq.

Venipuncture. 3 ml/kg of blood (to a maximum of 40 cc) were drawn after a 2 hour fast at the General Clinical Research Center at KUMC between 10 am and 12 am as previously described (Moddahl reference here). EMLA (topical lidocaine cream) was placed on the antecubital region of both arms and covered with tegaderm prior to blood draw. 10 ml of blood was collected in heparinized tubes on ice until processing within 15 minutes. This aliquot was centrifuged at 4° C. at 3000 g for 20 minutes, after which the plasma was stored in 1 ml aliquots at −80° C. until further processing, at which time it was transported on dry ice to the CMH/UMKC laboratory. The remaining blood (up to 30 cc) was placed on ice and transported within 30 minutes of blood draw to the same lab.

Extraction of oxytocin. A 1:1 mixture of sample and 0.1% trifluoroacetic acid (TFA) was mixed and centrifuged at 17000 g for 15 min at 4° C. to clarify the supernatant, which was saved and placed on ice. Hypersep C18 columns (500 mg; Fisher Scientific, Pittsburgh, Pa.) were positioned on a vacuum manifold and equilibrated with acetonitrile. Once equilibrated, 0.1% TFA was added. Sample supernatants were added to the column. Waste was discarded, and new collection tubes were placed in the manifold. Samples were eluted off the columns with 60:40 acetonitrile: 0.1% TFA. Samples were evaporated to dryness under vacuum, and stored at −20° C. until reconstitution.

Oxytocin Enzyme immunoassay (EIA). Oxytocin was assayed in the plasma using an oxytocin enzyme immunoassay kit (Assay Designs, Ann Arbor, Mich.) performed per manufacturer's instructions. The EIA kit was allowed to come to room temperature (RT) prior to start. Lyophilized samples were reconstituted with assay buffer (1:1 original sample volume: assay buffer). Standards were made according to manufacturer's instructions and used within 60 min of preparation. Standard amounts ranged from 12.5 to 1000 pg/ml. Samples and standards were run in duplicate. Assay buffer (100 μl) was placed in non-specific binding (NSB) and Bₒ wells, with 100 μl standards and samples placed in each well. Oxytocin conjugate (50 μl) was added to each well except blank and total activity (TA) wells. Oxytocin antibody (50 μl) was added to each well except blank, TA, and NSB. Samples incubated overnight (18-24 hr) at 4° C., and washed three times with wash buffer at RT. Wells were dried by firmly tapping the plate onto absorbent material. Oxytocin conjugate (5 μl) was added to TA wells, and p-Nitrophenyl Phosphate (pNPP) substrate (200 μl) was added to all wells and incubated for 1 hr at RT. Stop solution (50 μl) was added to each well, and samples were read at 405 nm with a correction between 570 and 590 nm on a PowerWaveₓ-I microplate spectrophotometer (Bio-Tek, Winooski, Vt.). Standard curves and concentrations were calculated using KC-4 software v.3.0 (Bio-Tek), Excel, and SigmaPlot 8.0.

PBMC isolation, hormone treatment and cell culture. PBMCs were isolated from heparinized blood using Histopaque 1077 (Sigma-Aldrich, St. Louis, Mo.). Briefly, 5 ml of blood was diluted 1:1 in PBS and carefully layered onto 3 ml of Histopaque 1077 in a 50 ml conical centrifuge tube. Tubes were centrifuged at 400 g for 30 min at RT. The buffy coat was removed from the tube and washed three times in complete RPMI without phenol red. Cell counts and viability were determined using Trypan blue. Cell viability was >95%. Resultant PBMC's were suspended in complete RPMI with 10% charcoal stripped, dilapidated fetal calf serum (FCS) (Invitrogen, Carlsbad, Calif.) and added in 1 ml aliquots to 12 well plate with vehicle and oxytocin (10 ⁻⁷M, 10 ⁻⁸M, and 10⁻⁹M) in RPMI+10% charcoal stripped, dilapidated FCS. Cells were incubated overnight (18-24 hr) before wells were harvested and rinsed. Cell suspensions were centrifuged at 500 g for 10 min at RT, and supernatant was aspirated. Buffer RLT Plus from the RNeasy Mini Plus Kit (QIAgen, Valencia, Calif.) was added to the cell pellets before vortexing to lyse cells. Samples were stored at −80° C. before RNA purification and isolation.

RNA isolation and quantitation. Samples were thawed, vortexed, and placed in QIAshredder columns. Columns were centrifuged for 2 min at 15000 g. The homogenized lysate was transferred to a gDNA Eliminator spin column and centrifuged for 30 sec at 9000 g. Equal volumes of 70% ethanol were added to the eluent. The mixture was transferred to RNeasy spin columns and centrifuged for 15 sec at 9000 g. Columns were washed with Buffer RW1 and the Buffer RPE, centrifuging for 15 sec at 9000 g, each time discarding the eluent. Buffer RPE was again added and columns centrifuged for 2 min at 9000 g to dry the column. Columns were then centrifuged for 1 min at 15000 g. RNA was eluted with the addition of Nuclease-free water and spun for 1 min at 9000 g. RNA was examined by spectrophotometry and quantitated using the Ribogreen assay (Invitrogen, Carlsbad, Calif.). Samples were diluted 1:200. Standards of 0, 20, 100, 500, and 1000 ng/ml were made from E. coli ribosomal RNA in TE buffer. Ribogreen reagent (1:200) was added to samples and standards, and samples were read on a Bio-Tek FLₓ800 fluorescent plate reader. RNA was diluted in nuclease-free water to a final concentration of 150 ng/ml cDNA.

cDNA construction and one-step real-time RT-PCR. Reverse transcription was performed using the SuperScript II Reverse Transcription Kit (Invitrogen, Carlsbad, Calif.). First, any residual DNA was digested away with DNase I for 15 min, with DNase activity subsequently quenched with 25 mM EDTA at 65° C. for 15 min. 10 mM dNTP and 0.5 ug/ml oligo (dT) were added to DNase-treated samples at 65° C. for 5 min. Samples were placed on ice, and reaction mixture containing DEPC-treated water, 25 mM MgCl₂, 10×RT buffer, 0.1 M dithiothreitol, and RNaseOUT recombinant RNase inhibitor was added at 42° C. After 2 min, SuperScript II Reverse transcriptase was added and incubated for 50 min at 42° C. Samples were cooled, and RNase H was added at 37° C. for 20 min. DEPC-treated water was added to make a final volume of 50 μl of 150 ng/ml cDNA.

One-step real-time RT-PCR was performed using the SYBR Green PCR kit (Bio-Rad, Hercules, Calif.). GAPDH and G protein sequences were obtained from the Gene Bank database and the following primers were constructed Using the GenBank sequence # NM_{—}080426: Gα_{S} sense: 5′-TCT ACC GGG CCA CGC ACC GC-3′ (SEQ ID NO. 1); Gα_{S} antisense: 5′-GCA GGA TCC TCA TCT GCT TC-3′ (SEQ ID NO. 2); Gα_{Q} sense: 5′-GAT GTT CGT GGA CCT GAA CC-3′ (SEQ ID NO. 3); Gα_{Q} antisense: 5′-CAA CTG GAC GAT GGT GTC CT-3′ (SEQ ID NO. 4); GAPDH sense: 5′-TGA CAA CTT TGG TAT CGT GGA AGG-3′ (SEQ ID NO. 5); GAPDH antisense: 5′-AGG GAT GAT GTT CTG GAG AGC C-3′ (SEQ ID NO. 6). A BLAST search was performed using the National Center for Biotechnology Information's BLAST WWW Server. RT-PCR was performed on the iCycler (Bio-Rad, Hercules, Calif.). The following parameters were used for the RT-PCR program: 95° C. at 3 min; 35 cycles of 95° C. at 20 sec, 56° C. at 20 sec, 72° C. for 20 sec; 95° C. for 1 min; and 55° C. at 1 min. Data were expressed as the ratio of the gene of interest compared to a housekeeping gene (GAPDH). That ratio was expressed as a percent of control. Control cDNA was created from a pool of PBMC's from healthy adult males and females and was used throughout the study.

### Results and Conclusions

After exposure to oxytocin, autistic males showed a more feminine pattern, meaning that mRNA for autistic males, after exposure to oxytocin, were more similarly related to levels of mRNA in control females, before exposure to oxytocin. In non-autistic patients mRNA levels went up after exposure to oxytocin, but this trend was not seen in the children with autism. There were sex differences for children with autism, as there was an upward trend in mRNA levels for females with autism, but this trend was not observed in males.

### Example 6

This example illustrates G-protein expression with PBMCs exposed to vasopressin and compares G-protein expression of patients with ASD and patients without ASD. Specifically, Gαs and Gαq expression was analyzed.

### Materials and Methods

The PBMCs utilized in Example 4 were used here to determine protein expression in patients with ASD and patients not exhibiting symptoms of ASD.

Membrane preparation for membrane-associated proteins. For protein studies, PBMC's were manipulated at 4° C., as previously described in Example 4. PBMC's were individually homogenized (1:30, wt/vol) in ice-cold 0.25 M sucrose and 50 mM Tris-HCL, (pH 7.6) in a Dounce homogenizer, centrifuged twice at 800×g for 5 min before centrifugation of the supernatants at 30,000×g for 60 min. The pellet was resuspended in assay buffer and used directly in the binding assay. Protein concentration was determined by BCA protein assay kit (Pierce, Rockford, Ill.) using bovine serum albumin (BSA) as a standard.

Immunoblot analysis for membrane-associated proteins. Total membrane proteins were prepared as for receptor assays above. Samples were electrophoresed in 12% Tris-HCl resolving gels and transferred to 0.45 μm nitrocellulose membranes (Bio-Rad, Hercules, Calif.) by electroblotting. Membranes were blocked in Tris-buffered saline with 0.1% Tween-20 (TBST) and 5% non fat dry milk at 25° C. for 1 h, then incubated overnight with affinity-purified monoclonal antibodies specific to G protein (described below) (Santa Cruz Biotech Inc., Santa Cruz, Calif.) in Tris-buffered saline with 0.1% Tween-20 (TBST) and 1% non fat dry milk. After three washes in TBST, membranes were incubated for 1 h in TBST and 1% non fat dry milk with horseradish peroxidase conjugated 2° antibodies (Jackson Immunoresearch, West Grove, Pa.). Bands were visualized by exposure of the membranes to autoradiographic film (MidSci, St. Louis, Mo.). The membranes were then stripped with Restore™ Western Blot Stripping Buffer (Pierce, Rockford Ill.) for 30 min at 37° C. and reprobed for actin protein as an internal control. The bands were quantified using a densitometer (GE Healthcare, Piscataway, N.J.).

Specific G protein antibodies. Specific Gαs and Gαq/11 antibodies made in rabbit without cross reactivity to other G proteins were used at a 1:500 dilution and originated from Santa Cruz Biotechnology (Santa Cruz, Calif.).

### Results and Conclusions

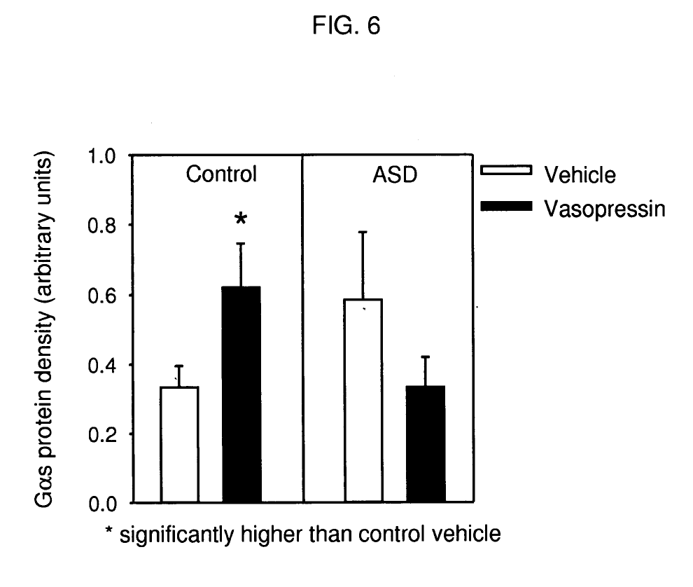

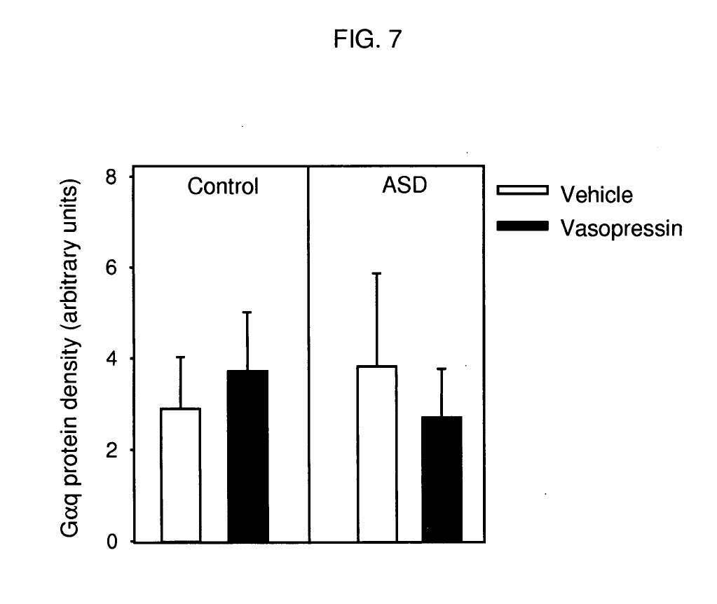

Patients with ASD exhibited higher levels of protein expression than controls. This result is consistent with the mRNA data and shows that the G-proteins are being translated, and that the upregulation of G-protein mRNA was not a compensatory mechanism for mRNA degradation. These results are illustrated in FIGS. 6 and 7 and Tables 14 and 15. As can be seen in FIG. 6, Gαs expression is much higher in the ASD group than the control with the control vehicle. This suggests that Gαs protein expression is higher in patients with ASD. FIG. 7 shows that Gαq expression is higher in patients with ASD when looking at the PBMCs in a control vehicle. For both G-proteins, Gαs and Gαq, vasopressin exposure causes a down-regulation trend in G-protein expression. This trend was not seen in patients without ASD, as protein expression appeared to be higher after vasopressin exposure. This suggests that down-regulators of G-protein expression, including vasopressin, may be a treatment option for patients with ASD, either when administered alone, or in combination with known therapies. G-protein levels could also be used as a diagnostic tool to indicate the presence of ASD in an individual. This diagnostic tool could be used alone or in combination with known diagnostic analyses

TABLE 14
Gαs protein expression after exposure to vasopressin
Gαsprotein

| | Control | Autism | Control | Autism |
|---|---|---|---|---|
| | Vehicle | Vehicle | Vasopressin | Vasopressin |
| Gαs protein | 0.28 | 0.78 | 0.51 | 0.52 |
| expression | 0.4 | 0.08 | 0.62 | 0.03 |
| | 0.27 | 0.6 | 0.67 | 0.7 |
| | 0.46 | 0.14 | 0.3 | 0.16 |
| | 0.28 | 0.01 | 0.83 | 0.03 |
| | 0.05 | 0.14 | 0.35 | 0.04 |
| | 0.83 | 0.04 | 1.63 | 0.01 |
| | 0.14 | 2.27 | 0.33 | 0.14 |
| | 0.19 | 1.37 | 0.19 | 0 |
| | 0.59 | 0.58 | 0.48 | 0.33 |
| | 0.16 | 0.37 | 0.29 | 0.69 |
| | 0.35 | 1.72 | 1.26 | 0.83 |
| | | 0.1 | | 0.33 |
| | | | | 0.86 |
| Average | .33 | .63 | .54 | .33 |

TABLE 15
Gαq protein expression after exposure to vasopressin
Gαqprotein

| | Control | Autism | Control | Autism |
|---|---|---|---|---|
| | Vehicle | Vehicle | Vasopressin | Vasopressin |
| Gαq protein | 0.81 | 0.04 | 2.86 | 0.19 |
| expression | 4.18 | 7 | 2.81 | 1.13 |
| | 10.59 | 8.79 | 13.29 | 0 |
| | 11.73 | 0.03 | 7.84 | 4.17 |
| | 0 | 0.02 | 0.25 | 12.58 |
| | 0 | 1.09 | 0.6 | 0 |
| | 0.09 | 0.22 | 0 | 0 |
| | 0 | 0.82 | 10.56 | 9.53 |
| | 2.42 | 25.99 | 0.26 | 0.36 |
| | 0 | 0.15 | 1.86 | 1.26 |
| | 0.39 | 0 | 3.96 | 4.64 |
| | 6.63 | 2.87 | 0.35 | 0 |
| | 0.96 | 3.06 | | 4.01 |
| | | | | 0 |
| Average | 2.9 | 3.88 | 3.72 | 2.71 |

### Example 7

This example illustrates G-protein expression with PBMCs exposed to oxytocin and compares G-protein expression of patients with ASD and patients without ASD. Specifically, Gαs and Gαq expression was analyzed.

### Materials and Methods

The PBMCs utilized in Example 5 were used here to determine protein expression in patients with ASD and patients not exhibiting symptoms of ASD.

Membrane preparation for membrane-associated proteins. For protein studies, PBMC's were manipulated at 4° C., as previously described in Example 4. PBMC's were individually homogenized (1:30, wt/vol) in ice-cold 0.25 M sucrose and 50 mM Tris-HCL, (pH 7.6) in a Dounce homogenizer, centrifuged twice at 800×g for 5 min before centrifugation of the supernatants at 30,000×g for 60 min. The pellet was resuspended in assay buffer and used directly in the binding assay. Protein concentration was determined by BCA protein assay kit (Pierce, Rockford, Ill.) using bovine serum albumin (BSA) as a standard.

Immunoblot analysis for membrane-associated proteins. Total membrane proteins were prepared as for receptor assays above. Samples were electrophoresed in 12% Tris-HCl resolving gels and transferred to 0.45 μm nitrocellulose membranes (Bio-Rad, Hercules, Calif.) by electroblotting. Membranes were blocked in Tris-buffered saline with 0.1% Tween-20 (TBST) and 5% non fat dry milk at 25° C. for 1 h, then incubated overnight with affinity-purified monoclonal antibodies specific to G protein (described below) (Santa Cruz Biotech Inc., Santa Cruz, Calif.) in Tris-buffered saline with 0.1% Tween-20 (TBST) and 1% non fat dry milk. After three washes in TBST, membranes were incubated for 1 h in TBST and 1% non fat dry milk with horseradish peroxidase conjugated 2° antibodies (Jackson Immunoresearch, West Grove, Pa.). Bands were visualized by exposure of the membranes to autoradiographic film (MidSci, St. Louis, Mo.). The membranes were then stripped with Restore™ Western Blot Stripping Buffer (Pierce, Rockford Ill.) for 30 min at 37° C. and reprobed for actin protein as an internal control. The bands were quantified using a densitometer (GE Healthcare, Piscataway, N.J.).

Specific G protein antibodies. Specific Gαs and Gαq/11 antibodies made in rabbit without cross reactivity to other G proteins were used at a 1:500 dilution and originated from Santa Cruz Biotechnology (Santa Cruz, Calif.).

### Results and Conclusions

The results for protein expression correlated with the mRNA data in Example 5. In non-autistic patients protein expression went up after exposure to oxytocin, but this trend was not seen in the children with autism. There were sex differences for children with autism, as there was an upward trend in protein expression for females with autism, but this trend was not observed in males.

### Example 8

This example will show up-regulators or agonists of vasopressin administered to patients with ASD normalizes G-protein levels and the patient will exhibit less symptoms associated with ASD, specifically, patients will exhibit less or fewer repetitive behaviors.

### Materials and Methods

The subjects described in Example 1 will be used here. All patients with ASD will have their G-protein levels evaluated as in the protocol of Example 6. The patients will be divided into two groups. Each patient in the experimental group will be administered a dose of a vasopressin agonist or up-regulator. The dose will be appropriate for the patient's age and size. The dose may be administered only once or may be administered several times over a given time period. G-protein levels will be evaluated in each patient in all groups at certain time points to determine their level of G-protein expression. Specifically, levels of Gαs and Gαq will be analyzed.

### Results and Conclusions

The ASD patients receiving the vasopressin agonist or up-regulator will show levels of Gαs and Gαq that are similar to those patients who do not have ASD, thereby meaning that their G-protein levels are normalized. In addition, repetitive behaviors will be diminished in patients receiving the agonist or up-regulator of vasopressin that had previously exhibited repetitive behaviors determined by any medically-accepted diagnostic for autism, including, but not limited to the ADOS and ADI. Further, patients receiving the vasopressin agonist or up-regulator will respond better to known therapy for autism than they had previously performed and in comparison to those patients not receiving a vasopressin agonist or up-regulator.

### Example 9

This example will show up-regulators or agonists of oxytocin administered to patients with ASD normalize G-protein levels and the patient will exhibit less or fewer symptoms associated with ASD, specifically, patients will exhibit less or fewer repetitive behaviors.

### Materials and Methods

The subjects described in Example 1 will be used here. All patients with ASD will have their G-protein levels evaluated as in the protocol of Example 7. The patients will be divided into two groups. Each patient in the experimental group will be administered a dose of an oxytocin agonist or up-regulator. The dose will be appropriate for the patient's age and size. The dose may be administered only once or may be administered several times over a given time period. G-protein levels will be evaluated in each patient in all groups at certain time points to determine their level of g-protein expression. Specifically, levels of Gαs and Gαq will be analyzed.

### Results and Conclusions

The ASD patients receiving the oxytocin agonist or up-regulator will show levels of Gαs and Gαq that are similar to those patients who do not have ASD. In addition, repetitive behaviors will be diminished in patients receiving the agonist or up-regulator of oxytocin that had previously exhibited repetitive behaviors determined by any medically-accepted diagnostic for autism, including, but not limited to the ADOS and ADI. Further, patients receiving the oxytocin agonist or up-regulator will respond better to known therapy for autism than they had previously performed and in comparison to ASD patients not receiving a oxytocin agonist or up-regulator.

### Example 10

This example will show down-regulators or antagonists of vasopressin administered to patients with ASD normalize G-protein levels and the patient will exhibit less or fewer symptoms associated with ASD, specifically, patients will exhibit less or fewer repetitive behaviors.

### Materials and Methods

The subjects described in Example 1 will be used here. All patients with ASD will have their G-protein levels evaluated as in the protocol of Example 6. The patients will be divided into two groups. Each patient in the experimental group will be administered a dose of a vasopressin antagonist or down-regulator, preferably, Tolvaptan (Otsuka America Pharmaceuticals), an oral vasopressin V₂-receptor antagonist. The dose will be appropriate for the patient's age and size. The dose may be administered only once or may be administered several times over a given time period. G-protein levels will be evaluated in each patient in all groups at certain time points to determine their level of g-protein expression. Specifically, levels of Gαs and Gαq will be analyzed.

### Results and Conclusions

The ASD patients receiving the vasopressin antagonist or down-regulator will show levels of Gαs and Gαq that are similar to those patients who do not have ASD. In addition, repetitive behaviors will be diminished in patients receiving the antagonist or down-regulator of vasopressin that had previously exhibited repetitive behaviors determined by any medically-accepted diagnostic for autism, including, but not limited to the ADOS and ADI. Further, patients receiving the vasopressin antagonist or down-regulator will respond better to known therapy for autism than they had previously performed and in comparison to ASD patients not receiving a vasopressin antagonist or down-regulator.

### Example 11

This example will show down-regulators or antagonists of oxytocin administered to patients with ASD show the patient to have normal G-protein levels and the patient will exhibit less symptoms associated with ASD, specifically, patients will exhibit less or fewer repetitive behaviors.

### Materials and Methods

The subjects described in Example 1 will be used here. All patients with ASD will have their G-protein levels evaluated as in the protocol of Example 6. The patients will be divided into two groups. Each patient in the experimental group will be administered a dose of an oxytocin antagonist or down-regulator, preferably, FE 200 440, an oxytocin antagonist. The dose will be appropriate for the patient's age and size. The dose may be administered only once or may be administered several times over a given time period. G-protein levels will be evaluated in each patient in all groups at certain time points to determine their level of G-protein expression. Specifically, levels of Gαs and Gαq will be analyzed.

### Results and Conclusions

The ASD patients receiving the oxytocin antagonist or down-regulator will show levels of Gαs and Gαq that are similar to those patients who do not have ASD. In addition, repetitive behaviors will be diminished in patients receiving the antagonist or down-regulator of oxytocin that had previously exhibited repetitive behaviors determined by any medically-accepted diagnostic for autism, including, but not limited to the ADOS and ADI. Further, patients receiving the oxytocin antagonist or down-regulator will respond better to known therapy for autism than they had previously performed and in comparison to those patients not receiving an oxytocin antagonist or down-regulator.

### REFERENCES

The teachings and content of the following references, as well as any others mentioned herein, are hereby incorporated by reference in their entireties.
1. Modahl C, Green L, Fein D, et al. Plasma oxytocin levels in autistic children. Biol Psychiatry. Feb. 15, 1998; 43(4):270-277.
2. Sweeten T L, Posey D J, McDougle C J. High blood monocyte counts and neopterin levels in children with autistic disorder. Am J Psychiatry. September 2003; 160(9): 1691-1693.
3. Croonenberghs J, Bosmans E, Deboutte D, Kenis G, Maes M. Activation of the inflammatory response system in autism. Neuropsychobiology. 2002; 45(1):1-6.
4. Jyonouchi H, Sun S, Itokazu N. Innate immunity associated with inflammatory responses and cytokine production against common dietary proteins in patients with autism spectrum disorder. Neuropsychobiology. 2002; 46(2):76-84.
5. Cook E H, Jr. Brief report: pathophysiology of autism: neurochemistry. J Autism Dev Disord. April 1996; 26(2):221-225.
6. Muhle R, Trentacoste S V, Rapin I. The genetics of autism. Pediatrics. May 2004; 113(5):e472-486.
7. Insel T R, O'Brien D J, Leckman J F. Oxytocin, vasopressin, and autism: is there a connection? Biol Psychiatry. Jan. 15, 1999; 45(2):145-157.
8. Ferguson J N, Aldag J M, Insel T R, Young Li. Oxytocin in the medial amygdala is essential for social recognition in the mouse. J. Neurosci. Oct. 15, 2001; 21(20):8278-8285.
9. Ferguson J N, Young L J, Hearn E F, Matzuk M M, Insel T R, Winslow J T. Social amnesia in mice lacking the oxytocin gene. Nat. Genet. July 2000; 25(3):284-288.
10. Gould T D, Manji H K. Signaling networks in the pathophysiology and treatment of mood disorders. J Psychosom Res. August 2002; 53(2):687-697.
11. Avissar S, Barki-Harrington L, Nechamkin Y, Roitman G, Schreiber G. Elevated dopamine receptor-coupled G(s) protein measures in mononuclear leukocytes of patients with schizophrenia. Schizophr Res. Jan. 15, 2001; 47(1):37-47.
12. Bezchlibnyk Y, Young L T. The neurobiology of bipolar disorder: focus on signal transduction pathways and the regulation of gene expression. Can J. Psychiatry. March 2002; 47(2):135-148.
13. Karege F, Buresi C, Golaz J, Schwald M, Malafosse A. Decreased expression of Galphas mRNA and protein levels in lithium-treated bipolar affective disorder. Hum Psychopharmacol. April 2000; 15(3):191-197.
14. Avissar S, Roitman G, Schreiber G. Differential effects of the antipsychotics haloperidol and clozapine on G protein measures in mononuclear leukocytes of patients with schizophrenia. Cell Mol. Neurobiol. December 2001; 21(6):799-811.
15. Uvnas-Mobcrg K, Bjokstrand E, Hillcgaart V, Ahlcnius S. Oxytocin as a possible mediator of SSRI-induced antidepressant effects. Psychopharmacology (Berl). February 1999; 142(1):95-101.
16. Lee R, Garcia F, van de Kar L D, Hauger R D, Coccaro E F. Plasma oxytocin in response to pharmaco-challenge to D-fenfluramine and placebo in healthy men. Psychiatry Res. May 30, 2003; 118(2):129-136.
17. Dolen G, Bear M F. Role for metabotropic glutamate receptor 5 (mGluR5) in the pathogenesis of fragile X syndrome. J. Physiol. Jan. 17, 2008.
18. Hagerman R J. Lessons from fragile X regarding neurobiology, autism, and neurodegeneration. J Dev Behav Pediatr. February 2006; 27(1):63-74.
19. Courchesne E, Carper R, Akshoomoff N. Evidence of brain overgrowth in the first year of life in autism. Jama. Jul. 16, 2003; 290(3):337-344.
20. Kenakin T. Receptors as microprocessors: pharmacological nuance on metabotropic glutamate receptors 1alpha. Sci STKE. Jul. 3, 2006; 2006(342):pc29.
21. Atkinson P J, Young K W, Ennion S J, Kew J N, Nahorski S R, Challiss R A. Altered expression of G(q/11alpha) protein shapes mGlu1 and mGlu5 receptor-mediated single cell inositol 1,4,5-trisphosphate and Ca(2+) signaling. Mol. Pharmacol. January 2006; 69(1):174-184.
22. Bourtchouladze R, Patterson S L, Kelly M P, Kreibich A, Kandel E R, Abel T. Chronically increased Gsalpha signaling disrupts associative and spatial learning. Learn Mem. November-December 2006; 13(6):745-752.
23. Kramer K M C B, Carter, S C, Wu J, Ottinger M A. Sex and species differences in plasma oxytocin using an enzyme immunoassay. Canadian Journal of Zoology. 2004; 82:1194-1200.
24. Cook E H. Autism: review of neurochemical investigation. Synapse. 1990; 6(3):292-308.
25. Hollander E, Bartz J, Chaplin W, et al. Oxytocin increases retention of social cognition in autism. Biol Psychiatry. Feb. 15, 2007; 61(4):498-503.
26. Hollander E, Novotny S, Hanratty M, et al. Oxytocin infusion reduces repetitive behaviors in adults with autistic and Asperger's disorders. Neuropsychopharmacology. January 2003; 28(1):193-198.
27. Kosfeld M, Heinrichs M, Zak P J, Fischbacher U, Fehr E. Oxytocin increases trust in humans. Nature. Jun. 2, 2005; 435(7042):673-676.
28. Kirsch P, Esslinger C, Chen Q, et al. Oxytocin modulates neural circuitry for social cognition and fear in humans. J Neurosci. Dec. 7, 2005; 25(49):11489-11493.

What is claimed is:

1. A diagnostic test for autism spectrum disorders, comprising an analysis of G-protein expression levels in a patient.

2. The diagnostic test of claim 1, wherein said G-protein is Gαs.

3. The diagnostic test of claim 1, wherein said G-protein is Gαq.

4. The diagnostic test of claim 1, wherein said G-protein levels are analyzed from birth to 30 months of age.

5. The diagnostic test of claim 1, wherein said diagnostic test comprises at least one factor in addition to said analysis.

6. The diagnostic test of claim 1, wherein said G-protein expression levels are at least 15% higher than in those patients without ASD.

7. The diagnostic test of claim 5, wherein said additional factor is a factor selected from a multifactorial test selected from the group consisting of Modified Checklist for Autism in Toddlers (M-CHAT), the Early Screening of Autistic Traits Questionnaire, and the First Year Inventory; the M-CHAT and its predecessor CHAT on children aged 18-30 months, Autism Diagnostic Interview (ADI), Autism Diagnostic Interview-Revised (ADI-R), the Autism Diagnostic Observation Schedule (ADOS) The Childhood Autism Rating Scale (CARS), and combinations thereof.

8. A method of diagnosing autism spectrum disorders, comprising the steps of
a. analyzing G-protein expression levels in a subject;
b. comparing said G-protein expression levels of the subject to those of individuals without an autism spectrum disorder; and
c. diagnosing autism spectrum disorders in the subject when the G-protein expression levels are higher than those of the individuals.

9. The method of claim 8, wherein said G-protein is Gαs.

10. The method of claim 8, wherein said G-protein is Gαq.

11. The method of claim 8, wherein said G-protein expression levels are at least 15% higher than in those patients without ASD.

12. The method of claim 8, wherein said G-protein expression levels are analyzed from birth to 30 months of age.

13. The method of claim 8, wherein said diagnostic test is used as one factor in a multifactorial test for autism spectrum disorders.

14. The method of claim 13, wherein said multifactorial test is selected from the group consisting of Modified Checklist for Autism in Toddlers (M-CHAT), the Early Screening of Autistic Traits Questionnaire, and the First Year Inventory; the M-CHAT and its predecessor CHAT on children aged 18-30 months, Autism Diagnostic Interview (ADI), Autism Diagnostic Interview-Revised (ADI-R), them Autism Diagnostic Observation Schedule (ADOS) The Childhood Autism Rating Scale (CARS), and combinations thereof.

15. A method of reducing repetitive behaviors in patients with autism spectrum disorders, comprising the step of administering a compound selected from the group consisting of oxytocin, an oxytocin agonist, an oxytocin antagonist, and combinations thereof, to a patient exhibiting repetitive behaviors.

16. The method of claim 15, wherein said oxytocin antagonist is FE 200 440.

17. A method of reducing repetitive behaviors in patients with autism spectrum disorders, comprising the step of administering a compound selected from the group consisting of vasopressin, a vasopressin agonist, a vasopressin antagonist, and combinations thereof, to a patient exhibiting repetitive behaviors.

18. The method of claim 17, wherein said vasopressin antagonist is Tolvaptan.

19. A method of reducing the symptoms of autism spectrum disorder, comprising the step of administering a compound to a patient with autism spectrum disorder which down-regulates expression of G-protein.

20. The method of claim 19, wherein said G-protein is Gαs.

21. The method of claim 19, wherein said G-protein is Gαq.

22. The method of claim 19, wherein said G-protein expression levels prior to down-regulation are at least 15% higher than in those patients without ASD.

23. The method of claim 19, wherein said G-protein expression levels are analyzed from birth to 30 months of age.

24. The method of claim 19, wherein said symptoms of autism spectrum disorder are selected from the group consisting of impairment in social interaction, impairment in social development, impairment with communication, behavior problems, repetitive behavior, stereotypy, compulsive behavior, sameness, ritualistic behavior, restricted behavior, self-injury, unusual response to sensory stimuli, impairment in emotion, problems with emotional attachment, impaired communication, and combinations thereof.

25. The method of claim 19, wherein said compound is vasopressin.

26. A method of reducing the symptoms of autism spectrum disorders, comprising the step of administering a compound selected from the group consisting of oxytocin, an oxytocin agonist, an oxytocin antagonist, vasopressin, a vasopressin agonist, a vasopressin antagonist, and combinations thereof to a patient with an autism spectrum disorder.

27. The method of claim 26, wherein said oxytocin antagonist is FE 200 440.

28. The method of claim 26, wherein said vasopressin antagonist is Tolvaptan.


SEQUENCE LISTING (6 sequences)

SEQ ID NO: 1 - DNA, 20 bp, Homo sapiens
tctaccgggccacgcaccgc

SEQ ID NO: 2 - DNA, 20 bp, Homo sapiens
gcaggatcctcatctgcttc

SEQ ID NO: 3 - DNA, 20 bp, Homo sapiens
gatgttcgtggacctgaacc

SEQ ID NO: 4 - DNA, 20 bp, Homo sapiens
caactggacgatggtgtcct

SEQ ID NO: 5 - DNA, 24 bp, Homo sapiens
tgacaactttggtatcgtggaagg

SEQ ID NO: 6 - DNA, 22 bp, Homo sapiens
agggatgatgttctggagagcc